(19) United States
(12) Patent Application Publication
(10) Pub. No.: US 2022/0079450 A1
(43) Pub. Date: Mar. 17, 2022

(54) Method and Probe System for Tissue Analysis in a Surgical Cavity in an Intraoperative Procedure

(71) Applicant: CytoVeris Inc., Farmington, CT (US)

(72) Inventors: David Fournier, Northborough, MA (US); Rishikesh Pandey, Unionville, CT (US); Michael A. Sapack, Southbury, CT (US); Patrick M. Curry, Glastonbury, CT (US); Alan Kersey, South Glastonbury, CT (US)

(21) Appl. No.: 17/478,513

(22) Filed: Sep. 17, 2021

Related U.S. Application Data
(60) Provisional application No. 63/079,738, filed on Sep. 17, 2020.

(51) Int. Cl. A61B 5/00

(52) U.S. Cl. CPC A61B 5/0091; A61B 5/0071; A61B 5/0075

(57) ABSTRACT

A system and method for determining the presence of cancerous tissue within a tissue cavity is provided. The system includes one or more excitation light sources, a photodetector, a probe, and a system controller. The probe includes an optically transparent probe body configured to fit within the tissue cavity. The system controller is in communication with the excitation light sources, the photodetector, and a memory storing instructions. The instructions when executed cause the system controller to a) control the excitation light sources to produce excitation light beams within the probe body, the excitation light beams operable to produce a response of the tissue to the interrogation and control the photodetector to detect the response and produce signals representative thereof; and b) produce information indicative of a presence of the cancerous tissue using the signals representative of the response.

This application claims priority to U.S. Patent Appln. No. 63/079,738 filed Sep. 17, 2020, which is hereby incorporated by reference in its entirety.

### BACKGROUND OF THE INVENTION

### 1. Technical Field

The present disclosure relates to devices and methods for assessing diseased tissue in general, and the devices and methods for assessing residual diseased tissue in an intraoperative procedure in particular.

### 2. Background Information

For many decades the reference method for the diagnosis of cancer has been histopathological examination of tissues using conventional microscopy. This process is known as surgical pathology. In surgical pathology, tissue samples can be produced from surgical procedures (tumor resection), diagnostic biopsies or autopsies. These tissue samples are subsequently subjected to a process that includes dissection, fixation, and cutting of tissue into precisely thin slices which are stained for contrast and mounted onto glass slides. The slides are examined by a pathologist under a microscope, and their interpretations of the tissue result in the pathology “read” of the sample.

Advanced optical and electromagnetic (“EM”) imaging approaches have been reported for the determination of tumor margin: These include the use of exogenous contrast-based fluorescence imaging [1, 2], near infrared spectroscopy [3], mass spectroscopy [4], terahertz reflectivity [5], Raman spectroscopy [6-12], hyperspectral imaging [13], autofluorescence life-time imaging [14], and the like.

Of these approach types, those that do not require any exogenous dye or contrast agents are particularly appealing in an in-vivo setting. Optical spectroscopy, in particular, offer significant advantages to patients by avoiding potential toxicological issues, Food and Drug Administration approval of the contrast agents as drugs, the cost of the contrast agents and increased surgical time associated with administering imaging agents.

The endogenous fluorescence signatures offer useful information that can be mapped to the functional, metabolic and morphological attributes of a biological specimen, and have therefore been utilized for diagnostic purposes. Biomolecular changes occurring in the cell and tissue state during pathological processes and disease progression result in alterations of the amount and distribution of endogenous fluorophores and form the basis for classification. Tissue autofluorescence has been proposed to detect various malignancies including cancer by measuring either differential intensity or lifetimes of the intrinsic fluorophores. Biomolecules such as tryptophan, collagen, elastin, nicotinamide adenine dinucleotide (NADH), flavin adenine dinucleotide (FAD), porphyrins, and the like present in tissue provide discernible and repeatable autofluorescence spectral patterns.

Raman spectroscopy in particular has been shown to provide a strong discrimination capability between normal and cancerous tissue. The spectral signatures observed using Raman spectroscopy arise due to the excitation of vibrational frequencies that are specific to a given molecular chemical bond, and the optical spectrum observed in what is known as the Raman fingerprint region (e.g., the wavenumber range 400-1800 cm′) provides a unique spectral fingerprint to identify different molecules and compounds. This has been used to assess the “margin status” of excised lump specimens in lumpectomy breast cancer surgery. In this form of surgery, often also referred to as “breast conserving surgery” or “BCS”, the surgeon is often challenged to remove all the cancerous tissue but also leave as much normal tissue in the breast as possible. This often results in what are referred to as positive margins of the excised specimen; i.e., indications of cancerous tissue either on, or in close proximity to, the surface of the excised specimen, meaning there this a very high likelihood of cancerous tissue remaining in the patient. This would manifest itself as residual cancerous tissue in the facets of the surgical cavity site remaining after the removal of the specimen.

While analysis of the excised specimen serves as a good “surrogate” for directly assessing the surgical cavity, a more direct approach is preferable considering soft and deformable lumpectomy excised samples whose orientation and geometry does not accurately correspond to the location of residual cancer in the cavity wall. The detection of cancer in the surgical cavity obviates the need for registration of the margin surface to the patient's body and also provides real-time feedback to the surgeons. To this end, handheld fiber probe-based approaches have been proposed. However, they are limited in area coverage and do not provide the assessment of the entire cavity. Consequently, there is a need to have a wide area, automated and adjustable probe which can provide the assessment of the surgical cavity.

### SUMMARY

According to an aspect of the present disclosure, a method of determining the presence of cancerous tissue within tissue that defines a tissue cavity is provided. The method includes: a) providing a probe having an optically transparent probe body configured to fit within the tissue cavity, the probe in communication with one or more excitation light sources and configured to direct an excitation light from the one or more excitation light sources to be incident with the tissue; b) using the probe to interrogate the tissue defining the tissue cavity with the excitation light at one or more wavelengths transmitted through the optically transparent probe body; c) detecting at least one response of the tissue through the optically transparent probe body to the interrogation and producing signals representative of the at least one response; and d) determining a presence of said cancerous tissue using the signals representative of the response.

In any of the aspects or embodiments described above and herein, the one or more wavelengths are operable to produce Raman scattering from a biochemical associated with the cancerous tissue.

In any of the aspects or embodiments described above and herein, the one or more wavelengths are operable to produce autofluorescence emissions from a fluorophore associated with the cancerous tissue.

In any of the aspects or embodiments described above and herein, the one or more wavelengths are operable to produce diffuse reflectance signals associated with the cancerous tissue.

In any of the aspects or embodiments described above and herein, the at least one response of the tissue to the interrogation may be diffuse reflectance of the excitation light, or Raman scattering, or autofluorescence emission, or any combination thereof.

In any of the aspects or embodiments described above and herein, the step of using the probe to interrogate the tissue defining the tissue cavity may include scanning substantially all of the tissue defining the tissue cavity with the excitation light.

In any of the aspects or embodiments described above and herein, the scanning may produce the at least one response from a plurality of regions of the tissue defining the tissue cavity.

In any of the aspects or embodiments described above and herein, the step of using the probe to interrogate the tissue defining the tissue cavity may include mapping the at least one response from the plurality of regions of the tissue defining the tissue cavity, the mapping including tissue cavity locational information associated with the at least one response from the respective tissue region.

According to another aspect of the present disclosure, a system for determining the presence of cancerous tissue within tissue that defines a tissue cavity is provided. The system includes one or more excitation light sources, one or more photodetectors, a probe, and a system controller. The probe includes an optically transparent probe body configured to fit within the tissue cavity, and the probe is in communication with the one or more excitation light sources and the one or more photodetectors. The system controller is in communication with the one or more excitation light sources, the one or more photodetectors, and a non-transitory memory storing instructions. The instructions when executed cause the system controller to: a) control the one or more excitation light sources to produce one or more excitation light beams within the probe body, the one or more excitation light beams operable to produce at least one response of the tissue to the interrogation, and control the one or more photodetectors to detect the at least one response and produce signals representative of the at least one response; and b) produce information indicative of a presence of the cancerous tissue using the signals representative of the response.

In any of the aspects or embodiments described above and herein, the one or more wavelengths may be operable to produce Raman scattering from one or more biochemicals associated with said cancerous tissue, or to produce autofluorescence emissions from a fluorophore associated with said cancerous tissue, or both.

In any of the aspects or embodiments described above and herein, the one or more wavelengths may be operable to produce diffuse reflectance signals associated with said cancerous tissue.

In any of the aspects or embodiments described above and herein, the probe may be configured to interrogate the tissue defining the tissue cavity by scanning substantially all of the tissue defining the tissue cavity with the excitation light.

In any of the aspects or embodiments described above and herein, the probe may include an optical scanning assembly that is disposed within the optically transparent probe body and the optical scanning assembly may be controllable to direct the one or more excitation light beams to be incident with a plurality of different regions of the tissue defining the tissue cavity and to transfer the at least one response from each region to the one or more photodetectors.

In any of the aspects or embodiments described above and herein, the probe body may have an at-rest geometric configuration that has a first interior cavity volume, and the probe body may be configured to be substantially conformable with the tissue cavity.

In any of the aspects or embodiments described above and herein, the probe body may have a first geometric configuration having a first geometric interior cavity volume, and the probe body may be selectively configurable in at least one second geometric configuration that has a second interior cavity volume, and the second interior cavity volume is larger than the first interior cavity volume.

According to another aspect of the present disclosure, a tissue cavity probe is provided that includes a stem, a probe body, an optical scanning assembly, and at least one light conduit. The probe body is connected to the stem. The probe body has at least one optically transparent wall that defines an interior cavity of the probe body. The optical scanning assembly is disposed within the interior cavity of the probe body. The at least one light conduit is configured to be connected to one or more light sources and in communication with the optical scanning assembly. The optical scanning assembly is configured to direct one or more beams of excitation light to be incident with a tissue defining at least part of a tissue cavity, and the probe is configured to collect at least one response from the tissue defining the at least part of the tissue cavity and transfer the at least one response to the at least one light conduit.

In any of the aspects or embodiments described above and herein, the optical scanning assembly may be controllable to direct the one or more excitation light beams to be incident with a plurality of different regions of the tissue defining the at least part of the tissue cavity.

The foregoing features and elements may be combined in various combinations without exclusivity, unless expressly indicated otherwise. These features and elements as well as the operation thereof will become more apparent in light of the following description and the accompanying drawings. It should be understood, however, the following description and drawings are intended to be exemplary in nature and non-limiting.

### BRIEF DESCRIPTION OF THE DRAWINGS

FIG. 1 is a diagrammatic illustration of an intra surgical cavity probe embodiment of the present disclosure.

FIG. 2 is a diagrammatic illustration of an intra surgical cavity probe embodiment of the present disclosure.

FIG. 3 is a diagrammatic illustration of an intra surgical cavity probe embodiment of the present disclosure, illustrating excitation light scanning and light detection.

FIG. 4 is a diagrammatic illustration of an intra surgical cavity probe embodiment of the present disclosure, illustrating excitation light scanning and light detection.

FIG. 5 is a schematic representation of an autofluorescence system portion of the present disclosure system.

FIG. 6 is a schematic representation of a Raman spectroscopic system portion of the present disclosure system.

FIG. 7 is a graph illustrating signal intensity versus wavenumber, including a bottom portion showing unfiltered Raman spectrum with wavenumber peaks and an upper portion showing filtered Raman signals at the wavenumber peaks.

### DETAILED DESCRIPTION

Aspects of the present disclosure include a novel and unobvious system 20 that utilizes one or more optical spectroscopic techniques such as Raman spectroscopy, tissue autofluorescence (AF), or diffuse reflectance, or near-infrared (NIR) absorption, or the like to assess the status of tissue defining a tissue cavity for the presence of diseased tissue (e.g., cancerous tissue) following a resection procedure. Diffuse reflectance refers to reflected excitation light that may be detected in a diminished intensity due to absorption and/or scattering of the interrogating excitation light within the tissue. To facilitate the description herein, the present disclosure will be described as utilizing Raman spectroscopy and AF. The present disclosure is not, however, limited to Raman, AF or combination thereof. The present disclosure is applicable to almost any form of tissue resection surgery and provides particular utility for cancer tissue resection surgery.

The system 20 includes at least one intra surgical cavity probe (“ISC probe 22”), at least one excitation light unit 24, at least one narrow band pass filter 26, at least one photodetector 28, and a system controller 30.

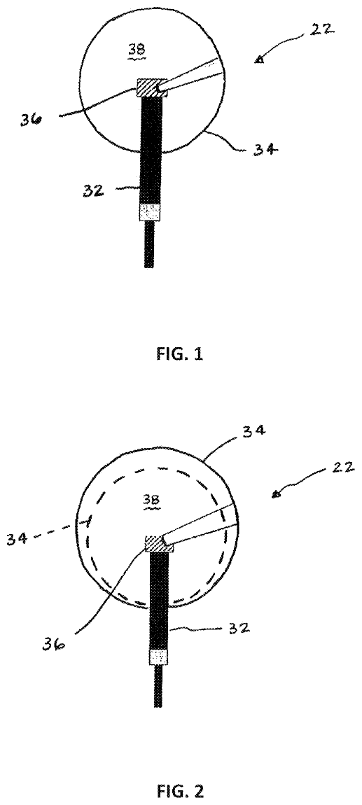

FIGS. 1 and 2 diagrammatically illustrate example ISC probe 22 embodiments that include a stem 32, a probe body 34, and an optical scanning assembly (OSA) 36. As will be described in greater detail below, the OSA 36 is connected to one or more optical fibers to deliver excitation light to and receive light from the OSA 36. The probe body 34 is connected directly or indirectly to the stem 32. The probe body 34 includes an interior cavity 38 defined by one or more walls, and the outer surface of the walls define the exterior surface of the probe body 34. The probe body 34 walls are optically transparent. The term “optically transparent” as used herein means that excitation light emitted within the interior cavity 38 can travel through the one or more walls at one or more wavelengths and at an intensity that is adequate for purposes of examination; i.e., examination using Raman spectroscopy, or autofluorescence (AF) emissions, or diffuse reflectance, or the like, and any combination thereof as described herein. Regarding Raman spectroscopic examination, the probe body 34 walls are configured to permit excitation light to pass through and to allow Raman scattering produced as a result thereof to pass into the probe body 34 for collection by the OSA 36. Raman scattering refers to inelastic scattering in a material where there is an exchange of energy between the incident photons and the vibrational energy levels of the molecular bonds present in the material. All materials exhibit Raman scattering in response to incident light. The Raman spectrum for a given material is typically complex due to the variety of molecular bonds present within the material, and the material is identifiable based on the Raman spectrum. An exemplary Raman spectrum may include a number of different peaks at a certain wavelengths or “wavenumber” offsets from the excitation light, which spectrum is uniquely characteristic of the material. Hence, the Raman spectrum of a particular material can be thought of as a “fingerprint” or “signature” of that particular material and can be used for identification purposes. Human tissue has a particularly, complex Raman spectrum, and the differences in the Raman spectrum associated with normal and diseased tissue can be subtle, but reproducible. Regarding AF examination, the probe body 34 walls are configured to permit excitation light to pass through and to allow fluorescently emitted light produced as a result thereof to pass into the probe body 34 for collection by the OSA 36. Tissue may naturally include certain fluorophores such as tryptophan, collagen, elastin, nicotinamide adenine dinucleotide (NADH), flavin adenine dinucleotide (FAD), porphyrins, and the like. Biomolecular changes occurring in the cell and tissue state during pathological processes and as a result of disease progression often result in alterations of the amount and distribution of these endogenous fluorophores. Hence, diseased tissues such as cancerous tissue, due to the marked difference in cell-cycle and metabolic activity can exhibit distinct intrinsic tissue AF emissions. Embodiments of the present disclosure may utilize these AF characteristics to identify regions of diseased tissue such as cancerous tissue.

In some embodiments, the probe body 34 may be configured to substantially maintain a predetermined geometry (e.g., spherical) and associated probe body volume. The term “probe body volume” as used herein refers to the volume occupied by the probe body 34 as defined by the exterior surface(s) of the probe body 34. In some embodiments, the probe body 34 may possess sufficient flexibility to permit deviation from an at-rest geometry to a geometry that at least partially conforms with one or more tissue surfaces within a tissue cavity being probed. The aforesaid conformity may permit more tissue cavity surface to be in contact with the probe body 34 than would be possible if the probe body 34 was substantially rigid. Moreover, a flexible probe body 34 may facilitate engagement with non-planar surfaces having deficits and peaks.

In some embodiments, the probe body 34 may be conformable from a first at-rest geometry to one or more second geometries that are expandable from a first geometry to a larger second geometry; e.g., the probe body volume of the first geometry is less than the probe body volume of the second geometry. In some of these embodiments, the probe body may be configured to permit substantially conformity with the tissue cavity being probed. FIG. 2 diagrammatically illustrates a probe body 34 that may be expanded from a first geometry to a larger second geometry; e.g., the probe body volume of the first geometry is less than the probe body volume of the second geometry. The expanded second geometry may adopt a variety of different geometric configurations to increase the amount of probe body wall in contact with tissue surface within a tissue cavity being probed. Non-limiting examples of an expandable probe body 34 include a probe body 34 that may be inflated, or a probe body 34 having one or more nested portions that can expand outwardly, or the like.

The volume of a surgically produced tissue cavity depends on the volume of the resected diseased tissue body. To accommodate a variety of different surgical cavity volumes, different embodiments of the ISC probe 22 may have different probe body volumes (initially and/or in unexpanded form, such as 2.0 cm, 2.5 cm, 3.0 cm diameters, etc.), or a given ISC probe 22 may be configured for use with a variety of different volume probe bodies; e.g., different volume probe bodies 34 can be selectively attached for use in different applications. In addition, the geometric shape of a surgical cavity may depend on the geometric shape of the resected tissue body. To accommodate a variety of different surgical cavity geometric shapes, different embodiments of the ISC probe 22 may have different probe body 34 geometries (initially and/or in unexpanded form, such as spherical, ellipsoid, etc.), or a given ISC probe 22 may be configured for use with a variety of different probe body 34 geometries; e.g., different probe bodies can be attached for use in different applications. Primary functions of an ISC probe include but are not limited to facilitation of a) illumination of surgical cavity tissue; b) collection of scattered/emitted light from the tissue after optical filtering; and c) scanning of the cavity so that the entire cavity wall can be sampled.

The OSA 36 may be disposed within the interior cavity 38 of the probe body 34 or may be insertable into the interior cavity 38 of the probe body 34 (e.g., storable within the stem 32, and displaceable from the stem 32 into the probe body 34 during use). The OSA 36 is configured to direct excitation light outwardly from the OSA 36 to the probe body wall and therethrough to the tissue of the interest. In some embodiments, the OSA 36 may be in communication with a light source 24 that is located remote from the OSA 36. In those embodiments that utilize a remote light source 24, a light pipe (e.g., one or more optical fibers, or a fiber optic cable) may be used as a conduit through which excitation light produced at a remote light source 24 can be transferred to the OSA 36. In some embodiments, an OSA 36 may include one or more light sources. The OSA 36 is configured to collect Raman scattering produced within the tissue of interest as a result of excitation light interrogation. In some embodiments, the OSA 36 may be in communication with one or more photodetectors 28 that are located remote from the OSA 36. In these embodiments, a light pipe (e.g., one or more optical fibers, or a fiber optic cable) may be used as a conduit through which the collected Raman scattering can be transferred from the OSA 36 to the remotely located one or more photodetectors 28. In some embodiments, the OSA 36 may be configured to collect native fluorescent light (e.g., AF) produced as a result of excitation light interrogation. Here again, a light pipe (e.g., one or more optical fibers, or a fiber optic cable) may be used as a conduit through which the collected AF light can be transferred from the OSA 36 to one or more photodetectors 28. The light pipe used to collect Raman scattering light (and/or the AF light) may be the same light pipe used to transfer excitation light from the light source(s) 24 to the optical scanning device, or a first light pipe may be used to transfer the excitation light to the OSA 36, and a second light pipe may be used to transfer collected Raman scattering light (and/or AF light) from the OSA 36 to the one or more photodetectors 28. In some embodiments, an OSA 36 may include one or more photodetectors. Hence, in some embodiments an OSA 36 may be configured to integrally produce excitation light and detect emitted light.

In some embodiments, an ISC probe 22 may not include an OSA 36. For example, an ISC probe 22 may alternatively include several optical fiber bundle probes, each having a portion disposed within the probe body 34 at different locations. The emitted/scattered/reflected light can be detected separately either using separate detectors or by using a single multichannel detector like CCD in communication with the optical fiber bundle probes. In this manner, a spectral map of the surgical cavity may be generated providing tissue/biochemical information and related locational information.

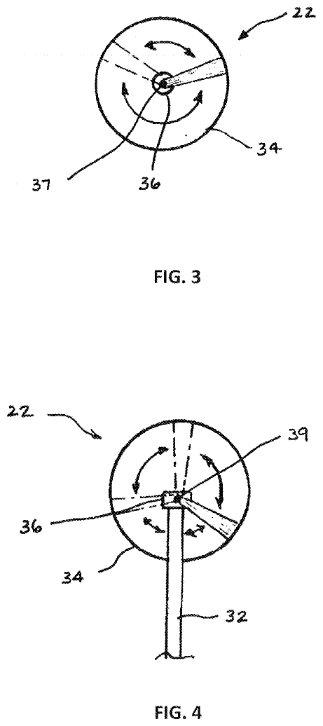

In some embodiments, the ISC probe 22 is configured such that the optical scanning device can direct excitation light in a substantial number of directions within the probe body 34 to enable a substantial amount of tissue of interest within the surgical cavity be exposed to excitation light. In this manner, the OSA 36 can be used to scan the surgical cavity with excitation light and collect Raman scattering, and/or AF emission, or other examination response signal and at the same time provide tissue cavity locational information associated with the collected Raman scattering and/or AF emissions (or other examination signal). In some embodiments, the OSA 36 may be connected to mechanical means (e.g., a rotation wheel or other mechanical structure) that permits the operator to rotate the OSA 36 (or a portion thereof) about an axis 37 that is parallel to the stem 32 during the scanning process; e.g., see FIG. 3. In other embodiments, the OSA 36 may include drive means (e.g., an electromagnetic drive motor) that can be controlled to rotate a portion or all of the OSA 36 for scanning purposes. In some embodiments, the OSA 36 may also be configured so that the angle at which the excitation light is emitted from the OSA 36 can be changed; e.g., rotated about an axis 39 that is perpendicular to the ISC probe stem 32 as shown in FIG. 4. Here again, the OSA 36 may be connected to a mechanical means or a powered means for rotating the OSA 36 during the scanning process. In those embodiments wherein the ISC probe 22 includes a powered element (e.g., to rotate the optical scanning device, etc.), the powered elements may be locally powered via a battery, or by a hardwire connection, or the like. Control of powered elements within the ISC probe 22 may be accomplished wirelessly or by hardwire. The present disclosure is not limited to any particular means for controlling the direction of the excitation light emitted from the OSA 36.

The excitation light unit 24 may include one or more LEDs, or one or more lasers, or may include a filtered source of white light (e.g., flash lamps), or any combination thereof. Each excitation light source within the excitation light unit 24 typically produce excitation light centered on particular wavelengths. The wavelengths produced by the excitation light unit 24 are typically chosen based on the photometric properties associated with one or more biochemicals or tissue types of interest; e.g., an excitation wavelength that causes desirable AF emissions, or Raman scattering, or diffuse reflectance, or the like, or any combination thereof from one or more biochemicals or tissue types of interest. Non-limiting examples of wavelengths that the light source 24 may be configured to produce include wavelengths of about 265 nm, 280 nm, 340 nm, and 365 nm, as these wavelengths may be useful with respect to certain biochemicals and/or tissue types present within a cancerous tissue sample. Embodiments of the present disclosure may include optical filtering elements configured to filter excitation light. Each optical filtering element is configured to pass a defined bandpass of wavelengths associated with an excitation light source and may take the form of a band pass filter. The excitation light filtering may take the form of an independent filtering element associated with each independent excitation light source, or a plurality of filtering elements disposed in a movable form (e.g., a wheel or a linear array configuration), or a single filtering element may be used for all excitation light sources, or each excitation light source may be configured to include a filtering element, or the like.

In some embodiments, the same excitation light unit 24 may be used for both fluorescence imaging and Raman spectroscopy (or diffuse reflectance) as those processes are described herein. In these embodiments, the excitation light unit 24 may be configured to produce excitation light within the sub-band of ultraviolet light between 200-280 nm (e.g., within the UVC sub-band of ultraviolet (UV) light, sometimes referred to as “deep ultraviolet light”). UV light at about 265 nm is particularly useful, as it produces high Raman scattering (e.g., 265 nm excitation produces about 77× the amount of Raman scattering that is produced using 785 nm excitation) and a Raman spectrum that is substantially fluorescence free. In addition, excitation light at 265 nm excites almost all native tissue chromophores and therefore its use may avoid the need to excite using a plurality of different excitation wavelengths. Examples of an acceptable light source 24 include UV LEDs and Nd-YAG lasers.

In some embodiments, a first excitation light unit (e.g., LEDs) may be used for fluorescence imaging and a second excitation light unit (e.g., lasers) may be used for Raman spectroscopy as those processes are described herein.

One or more photodetectors 28 may be used to detect Raman scattering and fluorescence emission (e.g., AF emissions) or reflectance signals from the tissue interrogated tissue with excitation light and produce signals representative thereof. The signals produced by the one or more photodetectors 28 are transferred to the system controller 30. Non-limiting examples of photodetectors 28 include photodetectors that convert light energy into an electrical signal such as a simple photodiode, or avalanche photodiodes, or other optical detectors of the type known in the art, such as CCD arrays, CMOS, ICCD, etc. As will be described below, some system 20 embodiments may include one or more first photodetectors 28 configured to detect fluorescence emissions from the interrogated tissue and produce signals representative thereof and one or more second photodetectors 28 to detect Raman scattering from the interrogated tissue and produce signals representative thereof.

The system controller 30 is in communication with other components within the system 20, such as the excitation light unit 24, the one or more photodetectors 28, and in some embodiments the ISC probe 22, and the like to control and/or receive signals therefrom to perform the functions described herein. The system controller 30 may include any type of computing device, computational circuit, processor(s), CPU, computer, or the like capable of executing a series of instructions that are stored in memory. The instructions may include an operating system, and/or executable software modules such as program files, system data, buffers, drivers, utilities, and the like. The executable instructions may apply to any functionality described herein to enable the system 20 to accomplish the same algorithmically and/or coordination of system 20 components. The system controller 30 includes or is in communication with one or more memory devices. The present disclosure is not limited to any particular type of memory device, and the memory device may store instructions and/or data in a non-transitory manner. Examples of memory devices that may be used include read-only memory, random access memory, volatile memory, non-volatile memory, static memory, dynamic memory, flash memory, cache memory, and/or any device that stores digital information. The system controller 30 may include, or may be in communication with, an input device that enables a user to enter data and/or instructions, and may include, or be in communication with, an output device configured, for example to display information (e.g., a visual display or a printer), or to transfer data, etc. Communications between the system controller 30 and other system 20 components may be via a hardwire connection or via a wireless connection.

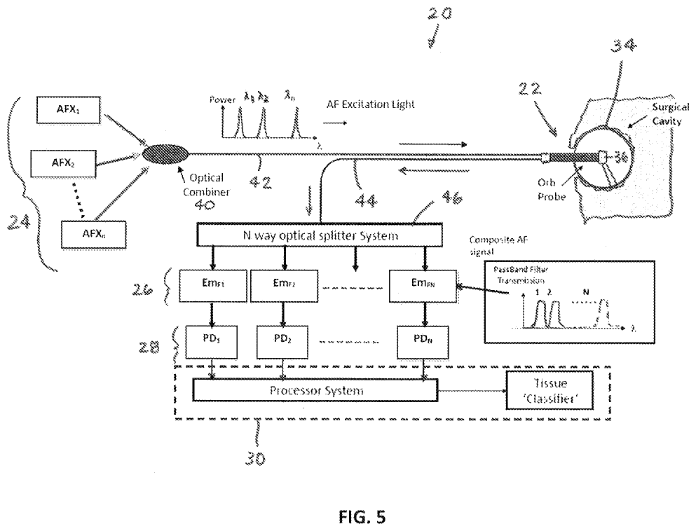

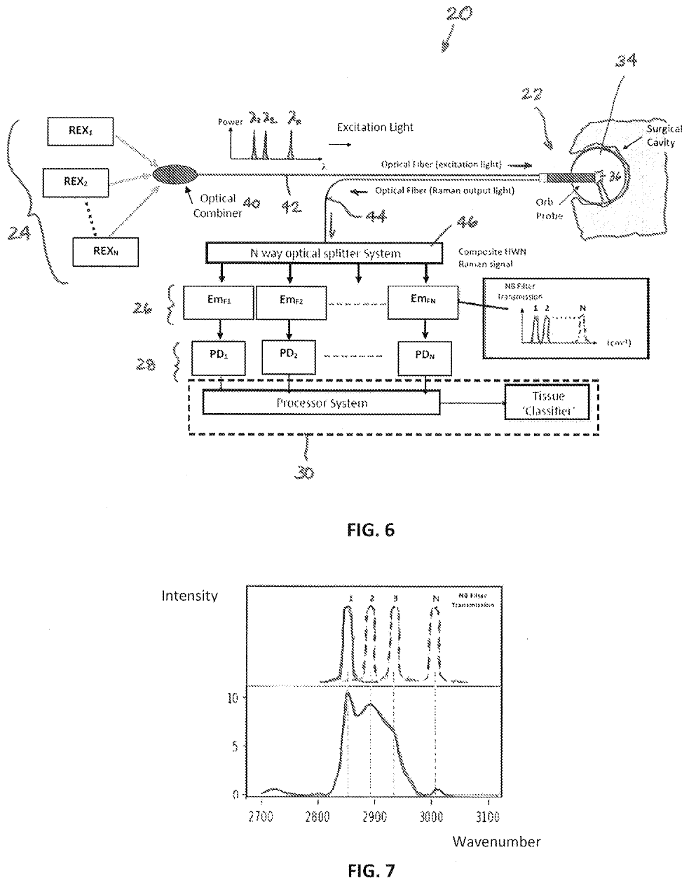

Aspects of the present disclosure utilize a form of hyperspectral detection. Some present disclosure system 20 embodiments combine both Raman spectroscopic sensing and tissue AF sensing to determine the presence/absence of residual cancerous within the tissue that defines a surgical cavity. An example of an AF sensing system portion is diagrammatically illustrated in FIG. 5 and a Raman spectroscopic system portion is diagrammatically illustrated in FIG. 6. The aforesaid system portions are shown independent of one another in FIGS. 5 and 6 to facilitate the explanation herein. In some embodiments, the Raman scattering and AF sensing system portions may be combined within a single system that includes a single ISC probe 22, or certain portions of each system portion may be common to both system portions. As will be clear from the description below, each of these system portions can be used to analyze tissue at a plurality (i.e., “N”) different wavelength/wavenumber windows using a plurality (i.e., “n”) excitation wavelengths, where “N” and “n” are integers that may or may not equal one another. As indicated herein, the present disclosure is not limited to using Raman spectroscopy and AF as optical techniques to assess the status of tissue defining a surgical cavity.

The exemplary AF system portion shown in FIG. 5 includes an excitation light unit 24 having a plurality of AF excitation light sources (e.g., AFX₁, AFX₂, . . . AFXₙ, where “n” is an integer greater than one), an optical combiner 40, one or more first optical fibers 42, an ISC probe 22, one or more second optical fibers 44, an optical splitter 46, a plurality of narrow band pass filters 26 (e.g., Em_{F1}, Em_{F2}, . . . Em_{FN}, where “N” is an integer greater than one), a plurality of photodetectors 28 (e.g., PD₁, PD₂, . . . PD_{N}), and a system controller 30. As stated above, the integers “n” and “N” may or may not equal one another. Each of the plurality of independent excitation light sources produce excitation light at a different wavelength. The respective excitation wavelengths are chosen based on native tissue fluorophores that may be present within diseased tissue and the significance of those fluorophores relative to diseased tissue. In other words, excitation wavelengths may be chosen that are known to produce identifiable AF emissions from native fluorophores having emission characteristics (e.g., intensity, density of signal within a given area, etc.) that provide information regarding the presence of diseased tissue (e.g., cancerous tissue).

The light produced by the AF excitation light unit 24 is passed to the optical combiner 40, and the optical combiner 40 in turn combines the respective light outputs into a composite beam of light. The one or more first optical fibers 42 transfer the composite beam of light from the optical combiner 40 to the ISC probe 22. The probe 22 is utilized to scan the tissue defining the interior of the surgical cavity with the combined beam of excitation light. The scanning involves applying excitation light to and collecting AF emissions from at least a portion of the tissue cavity, and typically substantially all of the tissue cavity. AF emissions caused by the excitation light interrogation of fluorophores within the tissue are collected by the OSA 36 and passed to the one or more second optical fibers 44 for transfer to the optical splitter 46. The optical splitter 46, in turn, splits the collected AF emissions into “N” portions (where “N” is an integer greater than 1) that pass into “N” channels. A respective narrow band pass filter 26 is disposed in each of the “N” channels to receive one of the collected AF emission portions. Each of the narrow band pass filters 26 may be centered at a wavelength in the UV/Visible/MR region, which wavelength is different from those of the other narrow band pass filters 26. The filtered AF emission output exiting each narrow band pass filter 26 is passed to a respective photodetector 28. Each photodetector 28 may be chosen to provide optimal performance at the wavelength of light passed by the narrow band pass filter 26, and at low intensity levels. In some embodiments, the light intensity monitored at each photodetector 28 can be integrated for a time duration (“T”) to increase the effective signal to noise ratio (“SNR”). Each respective photodetector 28 produces signals representative of the filtered AF emissions and those signals are communicated to the system controller 30.

The exemplary Raman spectroscopy system portion shown in FIG. 6 includes a plurality of Raman excitation light sources 24 (e.g., REX₁, REX₂, . . . REXₙ, where “n” is an integer greater than one), an optical combiner 40, one or more first optical fibers 42, an ISC probe 22, one or more second optical fibers 44, an optical splitter 46, a plurality of narrow band pass filters 26 (e.g., Em_{F1}, Em_{F2}, . . . Em_{FN}, where “N” is an integer greater than one), a plurality of photodetectors 28 (e.g., PD₁, PD₂, . . . PD_{N}), and a system controller 30. The integers “n” and “N” may or may not equal one another. Each of the plurality of independent Raman excitation light sources produce excitation light at a different wavelength. The respective Raman excitation wavelengths may be chosen based on one or more target biomolecules present in the diseased tissue of interest (which may vary depending on the type of diseased tissue; e.g., the type of cancerous tissue of interest). In other words, Raman excitation wavelengths may be chosen that are known to produce identifiable Raman scatterings that provide information regarding the presence of diseased tissue (e.g., cancerous tissue). As illustrated in FIG. 5, embodiments of the present disclosure system 20 allow the collected Raman spectrum to be sampled at discrete wavenumbers (e.g., wavenumbers 1, 2, 3 . . . N) within the high wavenumber (HWN) region of the Raman spectrum (2800-3800 cm′) corresponding to HWN peaks to facilitate signal processing.

The light produced by the Raman excitation light sources is passed to the optical combiner 40, and the optical combiner 40 in turn combines the respective light outputs into a composite beam of light. The one or more first optical fibers 42 transfer the composite beam of light from the optical combiner 40 to the ISC probe 22. The probe 22 is utilized to scan the tissue defining the interior of the surgical cavity with the combined beam of excitation light. Raman scatterings caused by the Raman excitation light interrogation of the tissue are collected by the OSA 36 and passed to the one or more second optical fibers 44 for transfer to the optical splitter 46. The optical splitter 46, in turn, splits the collected Raman scatterings into “N” portions (where “N” is an integer greater than 1) that pass into “N” channels. A respective narrow band pass filter 26 (Em_{F1} . . . Em_{FN}) is disposed in each of the “N” channels to receive one of the collected Raman scattering portions. Each of the narrow band pass filters 26 may be centered at a wavenumber, which wavenumber is different from those of the other narrow band pass filters 26. The present disclosure is not limited to using wavenumbers in any particular portion of the Raman spectrum; e.g., in some applications wavenumbers within the HWN region of the Raman spectrum may be used to facilitate signal processing. The filtered Raman scatterings output exiting each narrow band pass filter 26 is passed to a respective photodetector 28 (PD₁ . . . PD_{N}). In some embodiments, the Raman scattering intensity monitored at each photodetector 28 can be integrated for a time duration (“T”) to increase the effective signal to noise ratio (“SNR”). Each respective photodetector 28 produces signals representative of the filtered Raman scatterings and those signals are communicated to the system controller 30.

It should be noted that the present disclosure system 20 embodiments illustrated in FIGS. 5 and 6 are provided as diagrammatic non-limiting illustrations. System 20 embodiments may include various other system 20 components such as additional optical filters; e.g., to limit optical interference of other scattered light, or to direct Raman light to a detection path, or to block excitation light from a detection path, or any combination thereof.

In some embodiments, a single variable band or tunable band pass filter 26 and a single photodetector 28 may be used as an alternative to the multiple channel arrangement in the AF system portion, or in the Raman spectroscopic system portion, or both; i.e., the multichannel arrangement including “N” narrow band pass filters 26 and “N” photodetectors 28. For example, in the AF system portion the collected AF emissions may be passed to a tunable band filter 26 that can be controlled to sequentially filter the collected AF emissions at different bands, each centered at a different wavelength, and the sequentially filtered AF emission outputs then passed to a single photodetector 28 that produces signals representative of the respective filtered AF emissions, which signals are communicated to the system controller 30. In a similar fashion in regard to the Raman spectroscopic system portion, the collected Raman scatterings may be passed to a tunable band pass filter 26 that can be controlled to sequentially filter the collected Raman scatterings at different bands, each centered at a different wavenumber, and the sequentially filtered Raman scattering outputs may then be passed to a single photodetector 28 that produces signals representative of the respective filtered Raman scatterings, which signals are communicated to the system controller 30.

In some embodiments, a tunable excitation light unit 24 configured to selectively produce light at a plurality of different centered wavelengths as an alternative to the plurality of AF excitation light sources and optical combiner 40 described within the AF system portion, or the plurality of Raman excitation light sources and optical combiner 40 described within the Raman spectroscopic system portion, or both. In these alternative embodiments, the tunable excitation light unit 24 may be operated to sequentially produce each of the respective excitation wavelengths. In some embodiments, the excitation light sources within the AF system portion and the Raman spectroscopic system portion may be configured to include an optical switch rather than an optical combiner, and the respective excitation light sources may be operated to sequentially produce each of the respective excitation wavelengths. The operation of the present disclosure system 20 may include multiplexing techniques for producing the respective excitation wavelengths. The sequential use of different excitation wavelengths (“n”) allows for potentially “N”×“n” multispectral data points to be taken.

In some embodiments, the stored instructions within the system controller 30 may include an artificial intelligence/machine learning (AI/ML) algorithm trained classifier that includes a plurality of data sets that permit evaluation of tissue types based on input from the AF system portion and/or the Raman spectroscopic system portion. For example, some embodiments of the present disclosure may include an artificial intelligence/machine learning (AI/ML) algorithm that is trained to recognize AF emissions and Raman scatterings associated with different tissue types. In these embodiments, signals representative of AF emissions may be used within the AI/ML to perform an initial classification of tissue types, and the signals representative of the Raman scatterings may be used within the AI/ML algorithm to perform a subsequent classification of the tissue types identified as potentially cancerous in the initial classification. Data from the initial classification of the tissue types and the second Raman based classification may be used to provide an overall classification map of the sampled tissue locations. A dictionary learning, anomaly detector, convolutional neural network (CNN), or a random forrest type classifier are examples of algorithms that may be used. In some embodiments, an ensemble classifier that includes a plurality of base classifiers may be used. Alternatively, a plurality of base classifiers may be combined using a meta learner to get an optimum diagnostic performance. In some embodiments, various channel ratios can be used as inputs into a classifier. The present disclosure is not limited to these examples.

The combined use of Raman spectroscopy and another optical analysis technique (e.g., tissue AF) to ascertain the presence and location of residual cancerous tissue on or in proximity to the surface of a tissue cavity can provide several significant benefits. For example, the present disclosure may use combined Raman spectroscopy and AF sensing to determine the presence and location of cancerous tissue within the tissue defining the surgical cavity. Due to potentially broad and overlapping fluorescence spectral profiles of native biomolecules, in some instances the specificity of biomolecule identification using fluorescence spectroscopy may be limited. Nevertheless, fluorescence spectroscopy is useful to identify and locate cancerous tissue at a relatively fast speed. Raman spectroscopy, on the other hand, although having a slower measurement speed provides greater biochemical specificity and sensitivity. Combining the techniques provides confirmation of the presence and location of cancerous tissue, and greater specificity of and sensitivity to the types of cancer indicative biochemicals that may be present. As another example, as stated above the optical scanning device can be used to scan a surgical cavity with excitation light and produce a map indicating the presence and location of cancerous tissue on or in proximity to the surface of a tissue cavity. The presence/location information (e.g., map) can be used to not only verify whether any residual cancerous tissue is present, but also the location of the same; e.g., as an intraoperative procedure. If residual cancerous tissue is present, additional resection can be limited to the identified areas thereby minimizing or eliminating excess tissue resection. As yet another example, the present disclosure probe system may be readily adapted for use in a robotic/automated system.

While the principles of the disclosure have been described above in connection with specific apparatuses and methods, it is to be clearly understood that this description is made only by way of example and not as limitation on the scope of the disclosure. Specific details are given in the above description to provide a thorough understanding of the embodiments. However, it is understood that the embodiments may be practiced without these specific details.

It is noted that the embodiments may be described as a process which is depicted as a flowchart, a flow diagram, a block diagram, etc. Although any one of these structures may describe the operations as a sequential process, many of the operations can be performed in parallel or concurrently. In addition, the order of the operations may be rearranged. A process may correspond to a method, a function, a procedure, a subroutine, a subprogram, etc.

The singular forms “a,” “an,” and “the” refer to one or more than one, unless the context clearly dictates otherwise. For example, the term “comprising a specimen” includes single or plural specimens and is considered equivalent to the phrase “comprising at least one specimen.” The term “or” refers to a single element of stated alternative elements or a combination of two or more elements unless the context clearly indicates otherwise. As used herein, “comprises” means “includes.” Thus, “comprising A or B,” means “including A or B, or A and B,” without excluding additional elements.

It is noted that various connections are set forth between elements in the present description and drawings (the contents of which are included in this disclosure by way of reference). It is noted that these connections are general and, unless specified otherwise, may be direct or indirect and that this specification is not intended to be limiting in this respect. Any reference to attached, fixed, connected or the like may include permanent, removable, temporary, partial, full and/or any other possible attachment option.

No element, component, or method step in the present disclosure is intended to be dedicated to the public regardless of whether the element, component, or method step is explicitly recited in the claims. No claim element herein is to be construed under the provisions of 35 U.S.C. 112(f) unless the element is expressly recited using the phrase “means for.” As used herein, the terms “comprise”, “comprising”, or any other variation thereof, are intended to cover a non-exclusive inclusion, such that a process, method, article, or apparatus that comprises a list of elements does not include only those elements but may include other elements not expressly listed or inherent to such process, method, article, or apparatus.

While various inventive aspects, concepts and features of the disclosures may be described and illustrated herein as embodied in combination in the exemplary embodiments, these various aspects, concepts, and features may be used in many alternative embodiments, either individually or in various combinations and sub-combinations thereof. Unless expressly excluded herein all such combinations and sub-combinations are intended to be within the scope of the present application. Still further, while various alternative embodiments as to the various aspects, concepts, and features of the disclosures—such as alternative materials, structures, configurations, methods, devices, and components, and so on—may be described herein, such descriptions are not intended to be a complete or exhaustive list of available alternative embodiments, whether presently known or later developed. Those skilled in the art may readily adopt one or more of the inventive aspects, concepts, or features into additional embodiments and uses within the scope of the present application even if such embodiments are not expressly disclosed herein. For example, in the exemplary embodiments described above within the Detailed Description portion of the present specification, elements may be described as individual units and shown as independent of one another to facilitate the description. In alternative embodiments, such elements may be configured as combined elements.

### REFERENCES

The following references are hereby incorporated by reference in their respective entireties:
1. Nguyen and Tsien, “Fluorescence-guided surgery with live molecular navigation—a new cutting edge”, Nat Rev Cancer, 13(9), pp. 653-662, 2013.
2. Tummers, et al., “Real-time intraoperative detection of breast cancer using near-infrared fluorescence imaging and methylene blue”, Eur J Surg Oncol., 40(7), 850-858, 2014.
3. Dahr et al., “A diffuse reflectance spectral imaging system for tumor margin assessment using custom annular photodiode arrays”, Biomedical Optics Express, 3, (12), 2012.
4. Hanel et al., “Mass spectroscopy-based interoperative tumor diagnostics”, Future Sci OA, 5(3), 2019 March
5. Yaroslaysky. A, et al., “Delineating nonmelanoma skin cancer margins using terahertz and optical imaging”, J of Biomedical Photonics & Eng., 3(1), 2017.
6. Pence I., Mahadevan-Jansen A., “Clinical instrumentation and applications of Raman spectroscopy”, Chem Soc Rev.; 45 (7):1958-1979, 2016.
7. Talari, A. et al., “Raman Spectroscopy of Biological Tissues”, Applied Spectroscopy Reviews, 50:1, 46-111, 2015.
8. A. S. Haka, et al, “In vivo margin assessment during partial mastectomy breast surgery using Raman spectroscopy,” Cancer Res. 66(6), 3317-3322 (2006).
9. J. Mo, et al., “High wavenumber Raman spectroscopy for in vivo detection of cervical dysplasia,” Anal. Chem. 81(21), 8908-8915 (2009).
10. K. Lin, et al., “Optical diagnosis of laryngeal cancer using high wavenumber Raman spectroscopy,” Biosens. Bioelectron. 35(1), 213-217 (2012).
11. Aubertin et al., “Combining high wavenumber and fingerprint Raman spectroscopy for the detection of prostate cancer during radical prostatectomy”, 9, (9), Biomedical Optics Express, p. 4294, (2018)
12. R. Pandey et al, “Raman spectroscopy based molecular barcoding: realizing the value of high wavenumber region in breast cancer detection”, Proc. SPIE 11631, Advanced Biomedical and Clinical Diagnostic and Surgical Guidance Systems XIX, 1163105 (5 Mar. 2021).
13. Kho et al., “Hyperspectral Imaging for resection Margin Assessment during Cancer Surgery”, Clin Cancer Res; 25(12), Jun. 15, 2019.
14. Gorpas et al., “Autofluorescence lifetime augmented reality as a means for real-time robotic surgery guidance in human patients”, Sci Rep 9, 1187 (2019).

1. A method of determining the presence of cancerous tissue within tissue that defines a tissue cavity, comprising:
providing a probe having an optically transparent probe body configured to fit within the tissue cavity, the probe in communication with one or more excitation light sources and configured to direct an excitation light from the one or more excitation light sources to be incident onto the tissue;
using the probe to interrogate the tissue defining the tissue cavity with the excitation light at one or more wavelengths transmitted through the optically transparent probe body;
detecting at least one response of the tissue due to a tissue-light interaction through the optically transparent probe body to the interrogation and producing signals representative of the at least one response; and
determining a presence of said cancerous tissue using the signals representative of the response.

2. The method of claim 1, wherein the one or more wavelengths are operable to produce Raman scattering from one or more biochemical constituents associated with said cancerous tissue.

3. The method of claim 2, wherein the one or more wavelengths are operable to produce autofluorescence emissions from a fluorophore associated with said cancerous tissue.

4. The method of claim 1, wherein the one or more wavelengths are operable to produce autofluorescence emissions from a fluorophore associated with said cancerous tissue.

5. The method of claim 1, wherein the one or more wavelengths are operable to produce diffuse reflectance signals associated with said cancerous tissue.

6. The method of claim 1, wherein the at least one response of the tissue to the interrogation is diffuse reflectance of the excitation light, or Raman scattering, or autofluorescence emission, or any combination thereof.

7. The method of claim 1, wherein the step of using the probe to interrogate the tissue defining the tissue cavity includes scanning at least a portion of the tissue defining the tissue cavity with the excitation light.

8. The method of claim 7, wherein the scanning produces the at least one response from a plurality of regions of the tissue defining the tissue cavity.

9. The method of claim 8, wherein the step of using the probe to interrogate the tissue defining the tissue cavity includes mapping the at least one response from the plurality of regions of the tissue defining the tissue cavity, the mapping including tissue cavity locational information associated with the at least one response from the respective tissue region.

10. A system for determining the presence of cancerous tissue within tissue that defines a tissue cavity, comprising:
one or more excitation light sources;
one or more photodetectors;
a probe having an optically transparent probe body configured to fit within the tissue cavity, the probe in communication with the one or more excitation light sources and the one or more photodetectors; and
a system controller in communication with the one or more excitation light sources, the one or more photodetectors, and a non-transitory memory storing instructions, which instructions when executed cause the system controller to:
control the one or more excitation light sources to produce excitation light at one or more wavelengths within the probe body, the excitation light at the one or more wavelengths operable to produce at least one response of the tissue to the interrogation, and control the one or more photodetectors to detect the at least one response and produce signals representative of the at least one response; and
produce information indicative of a presence of said cancerous tissue using the signals representative of the response.

11. The system of claim 10, wherein the one or more wavelengths are operable to produce Raman scattering from one or more biochemical constituents associated with said cancerous tissue, or to produce autofluorescence emissions from a fluorophore associated with said cancerous tissue, or both.

12. The system of claim 10, wherein the one or more wavelengths are operable to produce diffuse reflectance signals associated with said cancerous tissue.

13. The system of claim 10, wherein the at least one response of the tissue to the interrogation is diffuse reflectance of the excitation light, or Raman scattering, or autofluorescence emission, or any combination thereof.

14. The system of claim 10, wherein the probe is configured to interrogate the tissue defining the tissue cavity by scanning substantially all of the tissue defining the tissue cavity with the excitation light.

15. The system of claim 10 wherein the probe includes an optical scanning assembly that is disposed within the optically transparent probe body and the optical scanning assembly is controllable to direct the one or more excitation light beams to be incident with a plurality of different regions of the tissue defining the tissue cavity and to transfer the at least one response from each region to the one or more photodetectors.

16. The system of claim 10, wherein the probe body has an at-rest geometric configuration that has a first interior cavity volume.

17. The system of claim 10, wherein the probe body is configured to be substantially conformable with the tissue cavity.

18. The system of claim 10, wherein the probe body has a first geometric configuration having a first geometric interior cavity volume, and the probe body is selectively configurable in at least one second geometric configuration that has a second interior cavity volume, and the second interior cavity volume is larger than the first interior cavity volume.

19. A tissue cavity probe, comprising;
a stem;
a probe body connected to the stem, the probe body having at least one optically transparent wall that defines an interior cavity of the probe body;
an optical scanning assembly disposed within the interior cavity of the probe body; and
at least one light conduit configured to be connected to one or more light sources and in communication with the optical scanning assembly;
wherein the optical scanning assembly is configured to direct one or more beams of excitation light to be incident with a tissue defining at least part of a tissue cavity, and the probe configured to collect at least one response from the tissue defining the at least part of the tissue cavity and transfer the at least one response to the at least one light conduit.

20. The probe of claim 19, wherein the at least one response is diffuse reflectance of the excitation light, or Raman scattering, or autofluorescence emission, or any combination thereof.

21. The probe of claim 19, wherein the optical scanning assembly is controllable to direct the one or more excitation light beams to be incident with a plurality of different regions of the tissue defining the at least part of the tissue cavity.

22. The probe of claim 19, wherein the probe body has an at-rest geometric configuration that has a first interior cavity volume, and the probe body is configured to be substantially conformable with the tissue cavity.

23. The probe of claim 19, wherein the probe body has a first geometric configuration having a first geometric interior cavity volume, and the probe body is selectively configurable in at least one second geometric configuration that has a second interior cavity volume, and the second interior cavity volume is larger than the first interior cavity volume.
